(19) United States
(12) Plant Patent Application Publication
(10) Pub. No.: US 2003/0009799 P1
(43) Pub. Date: Jan. 9, 2003

(54) 'AC-L' Acadie' strawberry

(76) Inventor: Shahrokh Khanizadeh, Baie d'Urfe (CA)

Correspondence Address: Therese Otis. P.Ag, AAFC-Horticultural R & D Center, 430 Gouin Blvd, St-Jean-sur-Richelieu, QC, J3B 3E6, CA

(21) Appl. No.: 09/888,415

(22) Filed: Jun. 26, 2001

(51) Int. Cl. A01H 5/00

(52) U.S. Cl. PLT/208

(57) ABSTRACT

A Nordic-type strawberry variety named ‘AC-L'Acadie’ combines the characteristics of adaptability to heavy soil conditions, and resistance to low winter temperature (<-30° C.), to herbicide terbacil and to leaf and root diseases as well as its high yield of large firm fruits with post-harvest fruit quality and integrity that enable storage and/or long distance shipping.

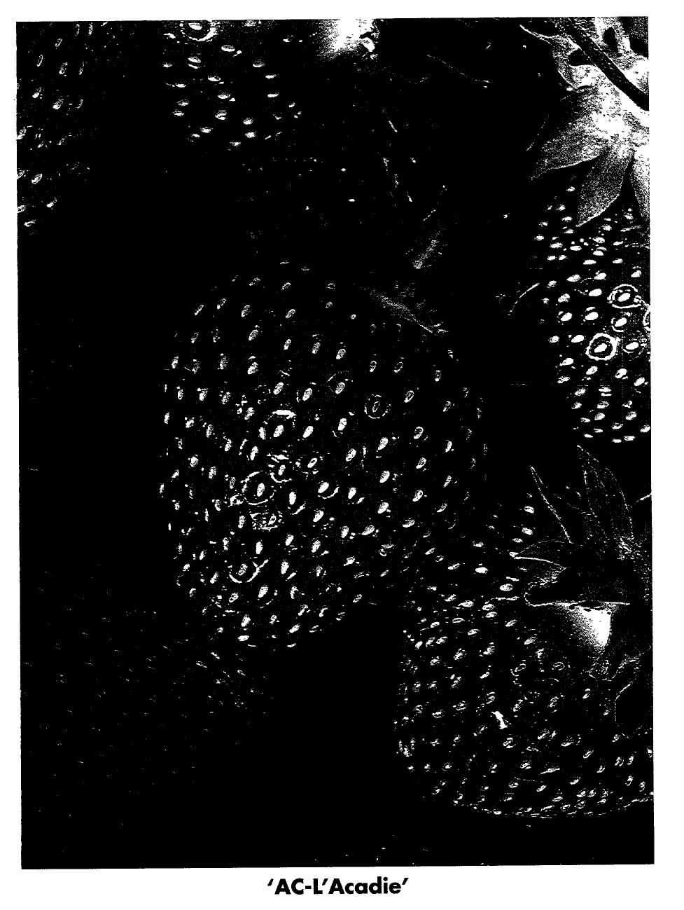

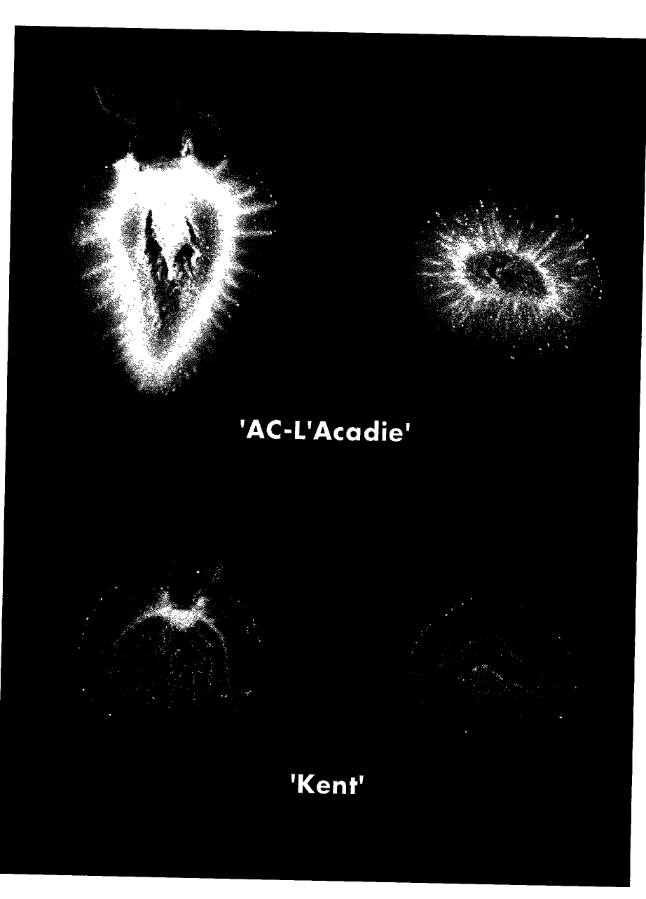

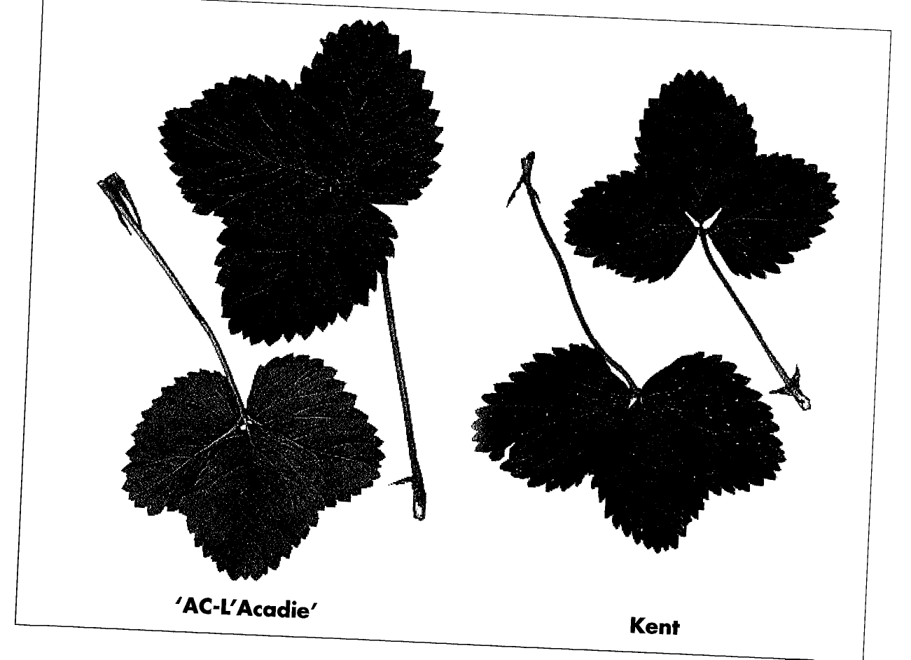

### BACKGROUND OF THE INVENTION

The invention relates to a new and distinctive cultivar of strawberry plant named ‘AC-L’Acadie’. This cultivar belongs to the genus Fragaria (×ananassa Dutch.) whose large shiny, pale red fruits are firm and store very well at room temperature for several days making them ideal for shipping or for growers who need to store fresh fruit. The cultivar is recommended for pick-your-own and/or fresh market producers.

The plants are moderately resistant to leaf diseases and partially resistant to the principal eastern races of red stele (Phytophthora fragariae Hickman).

Origin of the variety

The new cultivar ‘AC-L'Acadie’ was tested as SJ8916-50. It is a seedling resulting from a cross between ‘Glooscap’ and ‘Guardian’ made in 1989 by S. Khanizadeh. ‘Glooscap’ was used as a parent because of its high yields of high quality, glossy, dark red fruit with a reflexed calyx (Craig et al., 1991). ‘Guardian’ was used as a parent because of its large, firm, pale red fruit, and its known resistance to five races of red stele, powdery mildew (Sphaerotheca macularis Walls ex Fr.), leaf scorch (Diplocarpon earlina Ell. and Ev.), and verticillium wilt (Verticillium albo-atrum Reinke & Berth.) (Brooks and Olmo, 1997).

‘AC-L'Acadie’ was created in 1989 at Agriculture and Agri-Food Canada Horticultural R&D Centre, at St. Jean-sur-Richelieu, QC, Canada. It has been asexually reproduced by runners and tested at the same location since 1990, and at the Macdonald Campus of McGill University in Ste-Anne-de-Bellevue, QC, Canada, during the 1992-1994 fruiting seasons. It was also evaluated from 1996-1998 in controlled semi-commercial sites by our private partners in the province of Quebec, Canada, Lareault Inc., Les Fraises de l'lle d'Orleans Inc. and the Quebec Regional Small Fruit Trials of the Conseil des productions végétales du Québec (Provincial Agriculture Department Extension Office).

### DESCRIPTION OF THE FIGS.

1- Photo of fruits (2 sets).

2- Photo of sections of fruit compared to ‘Kent’ cultivar.

3- Photo of leaves (top and underside) compared to leaves of ‘Kent’ cultivar.

### DESCRIPTION OF THE VARIETY

‘AC-L'Acadie’ is a new June-bearing strawberry cultivar (Fragaria×ananassa Duch.) bred for Quebec and Eastern Central Canada growing conditions. ‘AC-L'Acadie’ produces large, firm fruit that store very well at room temperature for several days making them ideal for shipping or for growers who need to store fresh fruit. The plants are moderately resistant to leaf diseases and partially resistant to the principal eastern races of red stele (Phytophthora fragariae Hickman). The cultivar is recommended for pick-your-own and/or fresh market producers.

The prefix ‘AC’ in ‘AC-L'Acadie’ stands for Agriculture and Agri-Food Canada and ‘L’Acadie’ was chosen because this cultivar was originally selected at the Horticultural R&D Center substation located in the small town of L'Acadie where it was tested for several years. This is also the site where much of the strawberry research and breeding is being conducted in the province of Quebec.

### DETAILED BOTANICAL DESCRIPTION

Plants of ‘AC-L'Acadie’ have a medium density, are vigorous and have 4-7 inflorescences per crown. They can tolerate winter temperatures below -30° C. (with 10 cm straw mulch cover). The plants are globose in habit and of medium density. Petioles, which are of sparse pubescence and the hairs of which are upwards, are 12-22 cm long; the leaflets to the amount of three are longer than broad (1.25:1), large, dark green, flat, shiny, and obovate, each with about 17-24 serrations. The anthocyanin coloration of the stipules is medium. The leaves of ‘AC-L'Acadie’ are dark green in color; they are flat and weakly blistered. Inflorescence are positioned at foliage level with 10 to 14 flowers of medium size (flower petal length: 8.55 mm; petal width: 7.75 mm). The calyx diameter is larger than the corolla; the petals are broader than long and touching to overlapping. ‘AC-L’Acadie’ produces a medium number of stolons, which have medium thickness, pubescence and anthocyanin coloration.

‘AC-L'Acadie’ has semi-erect fruiting truss of medium length. ‘AC-L'Acadie’ produces large, longer than broad shiny, pale red fruit that ripen in midseason. Fruit shape is necked-conic and the semi-reflexed calyces decap as easily as those of ‘Chambly’ and ‘Glooscap’. There is a slight difference in shape between primary and secondary fruits. The fruit of even color do not show much unevenness of the surface with narrow to medium band without achenes. Achenes stand almost at surface level. The semi-reflexed calyx is smaller than the fruit, sets above fruit and has weak to medium adherence to fruit. The flesh of the fruit is lighter red than the skin and is sweet and firm with a fine texture and medium acidity. Fresh fruit store very well for up to 5 days at room temperature and frozen fruit retain their integrity after thawing.

### Performance and Diseases Resistance

Yields of ‘AC-L'Acadie’ over a four-year period are presented in Table 1 which shows that ‘AC-L’Acadie’ had yields similar to those of ‘Joliette’, ‘Oka’, ‘Kent’, ‘Glooscap’ and ‘Chambly’ during 1995-1998. The index of crop concentration of ‘AC-L'Acadie’ was very similar to that of ‘Joliette’, but more concentrated than that of ‘Kent’. No symptoms of powdery mildew were observed on ‘AC-L'Acadie’ in any of the sites during all the years of testing. ‘AC-L’Acadie’ had the same level of tolerance to leaf scorch, leaf blight (Dendrophoma obscurans Ell. and Ev.) and leaf spot (Mycosphaerella fragariae (Tul.) Lindau) as did ‘Honeoye’. No signs of gray mold fruit rot (Botrytis cinerea Pers. ex Fr.) were observed for any cultivars except ‘Glooscap’ which showed intermediate susceptibility. ‘AC-L’Acadie’ plants were ranked partially resistant to the six North American Eastern (NAE) races of red stele (A1 to A6) in a controlled environment (Khanizadeh et al., 1997). The plants are tolerant to the herbicide terbacil (5-chloro-3- (1,1 -d imethylethyl)-6-methyl-2,4-(1H ,3H)-pyrimidine-dione).

### Tests and Trials

‘AC-L'Acadie’ has been tested extensively at Agriculture and Agri-Food Canada, Horticultural R&D Centre substation located at L'Acadie, QC, Canada since 1989. The L'Acadie substation is located 35 km southeast of Montreal Island (lat. 45° N and 46 m elevation). Climate is characterized by extreme low temperatures in winter (<-25° C.); cool, wet, humid conditions in spring; and warm, dry, humid conditions (25° C. to 35° C., 70% RH) in summer. It has a clay loam soil with moderate to low drainage and little snow cover during the winter. ‘AC-L’Acadie’ has also been tested at McGill University, Ste-Anne de Bellevue (1995 to 1998) located at the far west of Montreal Island, QC (lat. 45° N and 27 m elevation) close to St. Louis Lake with a sandy clay soil, moderate drainage, and good snow cover; at Ministere de l'Agriculture des Pecheries et de l'Alimentation du Québec (MAPAQ), Sainte-Foy, QC (1992 to 1998) located in the west of Quebec city the capital of Quebec province (lat. 46° N and 90 m elevation) with sandy clay loam soil and extreme low temperature in winter; at lies d'Orleans, QC (1996 to 1999) located east of Quebec city the capital of Quebec province (lat. 46° N and 90 m elevation) with sandy clay loam soil and extreme low temperature in winter;; and at Lavaltrie, Quebec (from 1991 to 1993) located 35 km north of Montreal Island, QC (lat. 45° N and 20 m elevation) with sandy soil and a continental climate.

Adaptation and Uses

‘AC-L'Acadie’ is recommended for Eastern Central Canada, especially in areas where the climate is similar to that in Quebec Province. It performs very well in sandy soils and well in compact or heavy soils similar to those in L'Acadie where this genotype was first selected. ‘AC-L'Acadie’ is presently under evaluation in other provinces of Canada, in the United States, and in Europe. Because ‘AC-L'Acadie’ has good post-harvest fruit quality and integrity and stores well at room temperature, it is recommended for pick-your-own and/or fresh market producers. It is ideal for shipping or for growers who need to store fresh fruit.

TABLE 1
‘AC-L′Acadie’ total yield (g.m⁻¹ of row), fruit weight,
ripening season and indices of crop concentration
and earliness compared with those of other genotypes.

| | Total | | | Index of | |
|---|---|---|---|---|---|
| | yield | Wt./fruit | Ripening | crop | Index of |
| Genotype | (g.m⁻¹) | (g)^{y} | season^{x} | concentr. | earliness |
| ‘AC-L′Acadie’ | 2841 | 14.5 | M | 1.5 | 2.1 |
| Joliette | 2752 | 15.3 | M | 1.4 | 2.5 |
| Oka | 2653 | 13.6 | M | 1.0 | 3.0 |
| Kent | 2350 | 14.0 | M | 0.6 | 2.5 |
| Glooscap | 2322 | 11.8 | M | 1.3 | 2.5 |
| Chambly | 2286 | 13.9 | EM | 1.2 | 2.9 |
| Honeoye | 2226 | 11.8 | E | 1.1 | 3.0 |
| Jewel | 1837 | 13.9 | M | 1.1 | 1.6 |
| LSD_{0.05} | 600 | 2.3 | | 0.6 | 1.1 |

^{y}Averaged over 4 years (1995-1998), minimum of four replications per year, data from the L′Acadie site, genotypes are ranked by total yield.
^{x}M = midseason, EM = early-midseason, E = early.

TABLE 2
Firmness, flavor, skin color and shelf-life of
‘AC-L′Acadie’ compared with those of other genotypes.^{z}

| Genotypes | Firmness^{y} | Flavor^{y} | Skin color^{y} | Shelf-life^{x} |
|---|---|---|---|---|
| ‘AC-L′Acadie’ | 4.1 | 3.3 | 2.0 | 5.0 |
| Joliette | 4.2 | 3.3 | 2.8 | 4.0 |
| Oka | 2.0 | 3.7 | 4.0 | 1.0 |
| Kent | 3.0 | 3.0 | 2.2 | 2.0 |
| Glooscap | 2.7 | 3.1 | 3.6 | 1.0 |
| Chambly | 4.0 | 3.7 | 3.8 | 3.0 |
| Honeoye | 3.0 | 3.0 | 3.0 | 1.0 |
| Jewel | 4.0 | 3.0 | 2.6 | 3.0 |
| LSD_{0.05} | 0.1 | 0.2 | 0.2 | 1.3 |

^{z}Averaged over 4 years (1995-1998), minimum of four replications per year, data from the L′Acadie site.
^{y}Data were transformed to arcsin prior to analysis of variance (SAS Inst., 1988). Firmness: 1 = very soft, 5 = very firm. Flavor: 1 = poor, 5 = excellent. Skin color: 1 = very pale, 5 = dark red.
^{x}Number of day at room temperature for which the fruit were more than 95% marketable.

We claim:

1. A new and distinct cultivar of Fragaria×ananassa Duchh. plant named ‘AC-L'Acadie’, as herein shown and described, characterized particularly as to uniqueness by the combined characteristics of adaptability to heavy soil conditions, and resistance to low winter temperature (<-30° C.), to herbicide terbacil and to leaf and root diseases as well as its high yield of large firm fruits with post-harvest fruit quality and integrity that enable storage and/or long distance shipping.
